(19) United States
(12) Patent Application Publication
(10) Pub. No.: US 2017/0172416 A1
(43) Pub. Date: Jun. 22, 2017

(54) BIOLOGICAL INFORMATION ACQUISITION APPARATUS AND BIOLOGICAL INFORMATION ACQUISITION METHOD

(71) Applicant: SEIKO EPSON CORPORATION, Tokyo (JP)

(72) Inventors: Taki HASHIMOTO, Shiojiri-shi (JP); Ayae SAWADO, Kai-shi (JP)

(73) Assignee: SEIKO EPSON CORPORATION, Tokyo (JP)

(21) Appl. No.: 15/355,730

(22) Filed: Nov. 18, 2016

(30) Foreign Application Priority Data
Dec. 18, 2015 (JP) 2015-248013

(51) Int. Cl. A61B 5/00

(52) U.S. Cl. CPC A61B 5/0062; A61B 5/746; A61B 5/681

(57) ABSTRACT

A biological information acquisition apparatus includes a light emitting part provided on a detection surface facing a measurement site and irradiating the measurement site with a non-laser illumination light, a laser irradiation part provided on the detection surface and irradiating the measurement site with a laser, a light receiving part provided on the detection surface and receiving lights from the measurement site, and generating a first detection signal indicating light reception intensity when the illumination light is radiated and a second detection signal indicating light reception intensity when the laser is radiated, an irradiation control unit that controls whether or not to allow laser irradiation by the laser irradiation part according to the first detection signal, and an analytical processing unit that acquires biological information according to the second detection signal.

### BACKGROUND

1. Technical Field

The present invention relates to a technology for acquiring biological information.

2. Related Art

In measurement apparatuses that non-invasively measure biological information by laser irradiation of living organism, erroneous laser irradiation of retinae or the like is problematic. On the background, for example, Patent Document 1 (JP-A-2012-231978) discloses a configuration in which a contact condition between a living organism and the measurement apparatus is detected by a touch sensor and, if the contact condition is insufficient, laser irradiation is stopped. Further, Patent Document 2 (JP-A-2009-011593) discloses a configuration in which whether or not to allow laser irradiation is controlled according to a time difference between two peaks of a signal detected at laser irradiation.

However, in the technology of Patent Document 1, for example, there is a problem that, in the case where a foreign object intervenes between the living organism and the measurement apparatus, laser irradiation may be allowed even when the living organism and the measurement apparatus are actually separated. Further, in the technology of Patent Document 2, it is necessary to irradiate the living organism with a laser for a determination as to whether or not to allow laser irradiation, and the technology is insufficient for measures for preventing erroneous laser irradiation.

### SUMMARY

An advantages of some aspects of the invention is to reduce the possibility of erroneous irradiation with lasers used for acquisition of biological information.

A biological information acquisition apparatus according to a preferable aspect of the invention includes a light emitting part provided on a detection surface facing a measurement site and irradiating the measurement site with a non-laser illumination light, a laser irradiation part provided on the detection surface and irradiating the measurement site with a laser, a light receiving part provided on the detection surface and receiving lights from the measurement site, and generating a first detection signal indicating light reception intensity when the illumination light is radiated and a second detection signal indicating light reception intensity when the laser is radiated, an irradiation control unit that controls whether or not to allow laser irradiation by the laser irradiation part according to the first detection signal, and an analytical processing unit that acquires biological information according to the second detection signal. In the aspect, whether or not to allow the laser irradiation by the laser irradiation part is controlled according to the first detection signal indicating the light reception intensity when the illumination light is radiated by the light emitting part, and thereby, the biological information according to the second detection signal can be acquired with the reduced possibility of the erroneous laser irradiation.

In a preferable aspect of the invention, the analytical processing unit acquires biological information according to the first detection signal and biological information according to the second detection signal. In the aspect, the first detection signal is used for both the acquisition of the biological information and the control as to whether or not to allow the laser irradiation. Therefore, there is an advantage that the configuration of the biological information acquisition apparatus is simplified compared to a configuration in which a light emitting part for acquisition of the biological information and a light emitting part for control as to whether or not to allow the laser irradiation are separately provided.

In a preferable aspect of the invention, the first detection signal is a pulse wave signal containing a pulsation component of an artery of the measurement site, and the irradiation control unit controls whether or not to allow laser irradiation by the laser irradiation part according to a waveform of the first detection signal. In the aspect, whether or not to allow the laser irradiation is controlled according to the waveform of the first detection signal, and therefore, there is an advantage that whether or not to allow the laser irradiation may be appropriately controlled by the simple processing of analyzing the waveform of the first detection signal.

In a preferable aspect of the invention, the irradiation control unit allows laser irradiation by the laser irradiation part if a peak different from a first peak exists within a predetermined time from the first peak at which a signal value of the first detection signal becomes the maximum. In the aspect, whether or not to allow the laser irradiation is controlled according to whether or not the second peak exists within the predetermined time from the first peak, and therefore, the laser irradiation of the measurement site can be allowed only when the measurement site and the detection surface are in close contact to the degree that excludes the blood within capillaries.

In a preferable aspect of the invention, the irradiation control unit controls whether or not to allow laser irradiation by the laser irradiation part according to light reception intensity indicated by the first detection signal.

In the aspect, whether or not to allow the laser irradiation is controlled according to the light reception intensity indicated by the first detection signal, and therefore, there is an advantage that whether or not to allow the laser irradiation may be appropriately controlled by the simple processing of analyzing the signal intensity of the first detection signal.

In a preferable aspect of the invention, the irradiation control unit controls whether or not to allow laser irradiation according to intensity of a stationary component in the first detection signal. In the aspect, whether or not to allow the laser irradiation is controlled according to the intensity of the stationary component in the first detection signal, and therefore, laser irradiation to the measurement site can be allowed only when the measurement site and the detection surface are in close contact to the degree that excludes the blood within the capillaries.

A biological information acquisition apparatus according to another aspect of the invention includes a light emitting part provided on a detection surface facing a measurement site and irradiating the measurement site with a non-laser illumination light, a laser irradiation part provided on the detection surface and irradiating the measurement site with a laser, a light receiving part provided on the detection surface and receiving lights from the measurement site, and generating a first detection signal indicating light reception intensity when the illumination light is radiated and a second detection signal indicating light reception intensity when the laser is radiated, an irradiation control unit that controls whether or not to allow laser irradiation by the laser irradiation part according to light reception intensity of the light receiving part when the light emitting part is turned off, and an analytical processing unit that acquires biological information according to the first detection signal and the second detection signal. In the aspect, whether or not to allow the laser irradiation by the laser irradiation part is controlled according to the light reception intensity (external light intensity) of the light receiving part when the light emitting part is turned off, and thereby, biological information according to the first detection signal and the second detection signal can be acquired with the reduced possibility of erroneous laser irradiation.

A biological information acquisition method according to a preferable aspect of the invention by a biological information acquisition apparatus includes irradiating a measurement site with a non-laser illumination light from a detection surface facing the measurement site, controlling whether or not to allow laser irradiation to the measurement site according to a first detection signal indicating light reception intensity of a light received by a light receiving part provided on the detection surface when the illumination light is radiated, if the laser irradiation is allowed, irradiating the measurement site with a laser from a laser irradiation part provided on the detection surface, and acquiring biological information according to a second detection signal indicating light reception intensity of a light received from the measurement site by the light receiving part when the laser is radiated. In the aspect, whether or not to allow the laser irradiation by the laser irradiation part is controlled according to the first detection signal indicating the light reception intensity when the illumination light is radiated by the light emitting part, and thereby, biological information according to the second detection signal can be acquired with the reduced possibility of erroneous laser irradiation.

### BRIEF DESCRIPTION OF THE DRAWINGS

The invention will be described with reference to the accompanying drawings, wherein like numbers reference like elements.

FIG. 1 is a configuration diagram of a biological information acquisition apparatus according to a first embodiment of the invention.

FIG. 2 is a configuration diagram that exemplifies functions of the biological information acquisition apparatus.

FIG. 3 is a waveform chart of detection signals.

FIG. 4 is a waveform chart of differential values of the detection signals.

FIG. 5 is a flowchart of a biological information acquisition method.

FIG. 6 is a waveform chart of detection signals in the second embodiment.

FIG. 7 is an explanatory diagram of intensity of stationary components of the detection signals.

FIG. 8 is an explanatory diagram of light reception intensity by a light receiving part in the third embodiment.

### DESCRIPTION OF EXEMPLARY EMBODIMENTS

### First Embodiment

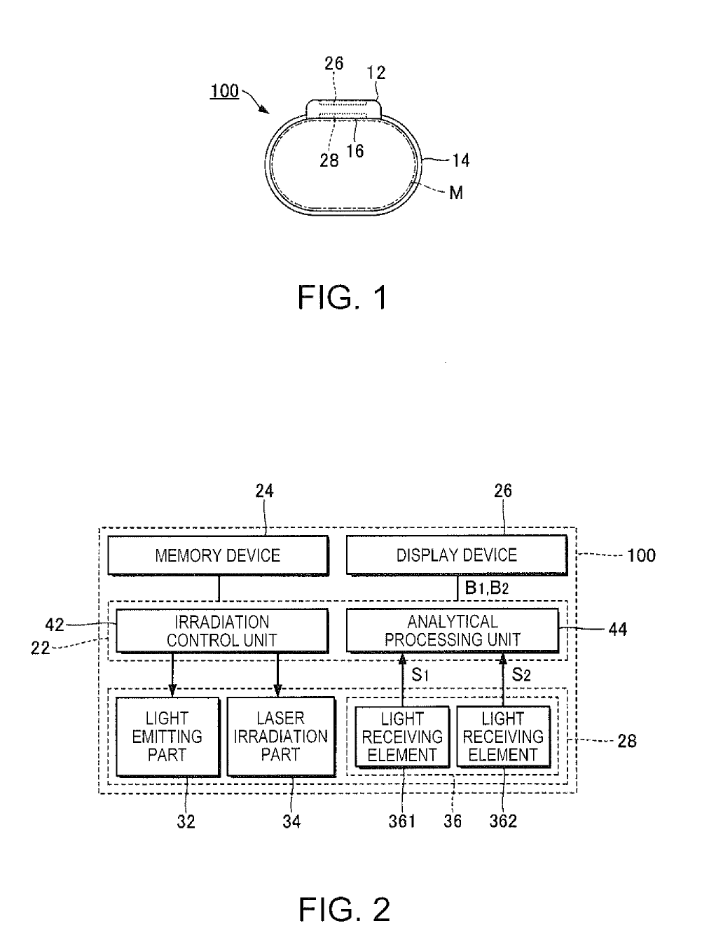

FIG. 1 is a side view of a biological information acquisition apparatus 100 according to a first embodiment of the invention. The biological information acquisition apparatus 100 of the first embodiment is a measurement apparatus that non-invasively acquires biological information of a subject (an example of a living organism). The biological information acquisition apparatus 100 of the first embodiment is a wristwatch-type apparatus including a casing part 12 and a belt 14, and held with the belt 14 wrapped around a wrist as an example of a site (hereinafter, referred to as “measurement site”) M as a measuring object of a body of the subject.

FIG. 2 is a configuration diagram with a focus on functions of the biological information acquisition apparatus 100 of the first embodiment. As exemplified in FIG. 2, the biological information acquisition apparatus 100 of the first embodiment includes a control device 22, a memory device 24, a display device 26, and a detection device 28. The control device 22 and the memory device 24 are provided inside of the casing part 12. As exemplified in FIG. 1, the display device 26 is provided on a surface of the casing part 12 (a surface opposite to the measurement site M), and the detection device is provided on a surface (hereinafter, referred to as “detection surface”) 16 facing the measurement site M of the casing part 12. The detection surface 16 is a flat surface or curved surface.

The detection device 28 in FIG. 2 is an optical sensor that generates detection signals S (S1, S2) according to a body condition of the subject, and includes a light emitting part 32, a laser irradiation part 34, and a light receiving part 36. The light emitting part 32 includes e.g. a light emitting device such as an LED (Light Emitting Diode), and can irradiate the measurement site M with light (hereinafter, referred to as “illumination light”) from a light emitting surface provided in the detection surface 16 of the casing part 12. On the other hand, the laser irradiation part 34 includes e.g. a semiconductor laser (LD: Laser Diode), and can irradiate the measurement site M with a laser from a light emitting surface provided in the detection surface 16 of the casing part 12.

The laser radiated by the laser irradiation part 34 is a light output through resonance by a resonator and traveling in a straight line coherently in a narrow range. The illumination light radiated by the light emitting part 32 is a non-laser light incoherent in a wider range than that of the laser. If a gap exists between the measurement site M and the detection surface 16, it maybe possible that the laser radiated by the laser irradiation part 34 leaks from the gap to the outside and erroneously irradiates the subject or the like (the subject or a person around). Therefore, it is important to allow the laser irradiation by the laser irradiation part 34 only when the detection surface 16 is in close contact with the measurement site M with no gap.

The light receiving part 36 in FIG. 2 generates detection signals S (S1, S2) according to amounts of received lights. The light receiving part 36 of the first embodiment includes a light receiving element 361 and a light receiving element 362. Each of the light receiving element 361 and the light receiving element 362 is a photoelectric conversion element (e.g. photodiode) that receives light by a light receiving surface provided in the detection surface 16 of the casing part 12. The light receiving element 361 generates the detection signal S1 that indicates the light reception intensity of a light coming from the measurement site M when the illumination light is radiated by the light emitting part 32 (i.e., a component passing through the measurement site M of the illumination light). On the other hand, the light receiving element 362 generates the detection signal 52 that indicates the light reception intensity of a light coming from the measurement site M when the laser is radiated by the laser irradiation part 34 (i.e., a component passing through the measurement site M of the laser radiated by the laser irradiation part 34). The detection signal S1 is an exemplification of a first detection signal and the detection signal S2 is an exemplification of a second detection signal. Note that, actually, an A/D converter that converts the detection signal S1 and the detection signal S2 from analog signals into digital signals is provided, however, the illustration thereof is omitted for convenience in FIG. 2. Further, the light receiving part 36 can be formed by a single light emitting device that receives both the illumination light radiated by the light emitting part 32 and the laser light radiated by the laser irradiation part 34.

The illumination light radiated by the light emitting part 32 and the laser radiated by the laser irradiation part 34 are transmitted through the epidermis of the measurement site M of the subject and reaches a blood vessel inside, partially absorbed by the blood within the blood vessel and scattered and transmitted within a living tissue, and output from the epidermis on the detection surface 16 side. The blood vessel of the measurement site M repeatedly expands and contracts in the same cycle as that of the heartbeat. The absorbance by the blood within the blood vessel differs between expansion and contraction, and the detection signals S generated by the light receiving part 36 are pulse wave signals containing periodic fluctuation components corresponding to a pulsation component (volume pulse wave) of the artery of the measurement site M.

The control device 22 in FIG. 22 is an arithmetic processing unit such as a CPU (Central Processing Unit) or FPGA (Field-Programmable Gate Array) that controls the entire of the biological information acquisition apparatus 100. The memory device 24 includes e.g. a nonvolatile semiconductor memory and stores programs to be executed by the control device 22 and various kinds of data to be used by the control device 22. The control device 22 of the first embodiment realizes a plurality of functions (irradiation control unit 42, analytical processing unit 44) for acquiring the biological information of the subject by executing the programs stored in the memory device 24. Note that a configuration in which the respective functions of the control device 22 are distributed in a plurality of integrated circuits or a configuration in which part or all of the functions of the control device 22 are realized by a dedicated electronic circuit may be employed.

The analytical processing unit 44 analyzes the detection signals (S1, S2) generated by the light receiving part 36, and thereby, acquires biological information B (B1, B2) of the subject. Specifically, the analytical processing unit 44 estimates the pulse beat of the subject by the analysis of the detection signal S1 as biological information B1 and estimates the blood flow velocity and the blood pressure of the subject by the analysis of the detection signal S2 as biological information B2. The biological information Bland the biological information B2 are different. The display device 26 in FIG. 2 is e.g. a liquid crystal display panel and displays the biological information B acquired by the analytical processing unit 44. Note that the biological information B (B1, B2) is not limited to the above described exemplification. For example, the concentrations of the body compositions of the properties in the blood (e.g. glucose concentration, hemoglobin concentration, oxygen concentration, neutral fat concentration) can be estimated as the biological information B.

The irradiation control unit 42 in FIG. 2 controls irradiation with light by the light emitting part 32 and the laser irradiation part 34. Specifically, the irradiation control unit 42 of the first embodiment allows the light emitting part 32 to radiate the illumination light and, on the other hand, controls whether or not to allow laser irradiation by the laser irradiation part 34 according to the detection signal S1 indicating the light reception signal when the illumination light is radiated.

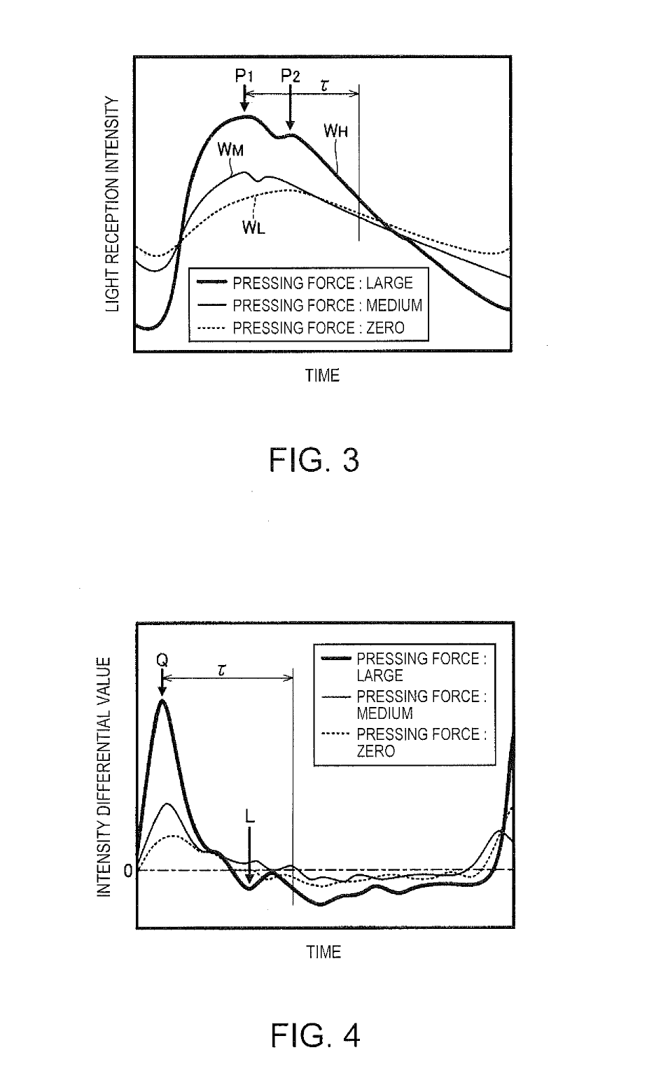

FIG. 3 is a waveform chart of single wavelength corresponding to a single heartbeat of the detection signal S1. Waveforms of the detection signal S1 are shown with respect to a plurality of cases in which the contact condition of the detection surface 16 with the measurement site M is varied. Specifically, the waveform WL in FIG. 3 is the waveform of the detection signal S1 in a state in which the detection surface 16 of the casing part 12 is simply mounted on the measurement site M of the subject (i.e., a state in which the pressing force acting from the detection surface 16 on the measurement site M is substantially zero). The waveform WH in FIG. 3 is the waveform of the detection signal S1 in a state in which the detection surface 16 of the casing part 12 is sufficiently pressed on the measurement site M of the subject (i.e., a state in which the pressing force acting from the detection surface 16 on the measurement site M is larger) Further, the waveform WM in FIG. 3 is the waveform of the detection signal S1 in a state in which the detection surface 16 of the casing part 12 is pressed on the measurement site M of the subject with a medium pressing force lower than that in the state of the waveform WH.

In a condition in which the waveform WL is observed, a gap may exist between the measurement site M and the detection surface 16, and the laser radiated from the laser irradiation part 34 may leak from the gap to the outside. In other words, it may be possible that the laser erroneously irradiates the subject or the like. On the other hand, in a condition in which the waveform WH is observed, the measurement site M and the detection surface 16 are sufficiently in close contact and the laser radiated from the laser irradiation part 34 does not leak to the outside. In other words, erroneous laser irradiation of the subject or the like can be avoided.

As understood from FIG. 3, in the waveform WH and the waveform WM in the state in which the measurement site M and the detection surface 16 are sufficiently in close contact, almost simultaneous two peaks (P1, P2) are observed. On the other hand, in the waveform WI in the state in which the measurement site M and the detection surface 16 are not sufficiently in close contact, only one peak is observed. That is, a tendency that the waveform of the detection signal S1 differs depending on the contact state (the degree of close contact) between the measurement site M and the detection surface 16 may be confirmed from FIG. 3. It is estimated that one reason that the above described waveform differences are observed is, as will be described in detail, the influence by reflected wave of the pulse wave in the respective sites of the body of the subject varies according to the condition of the capillaries in the measurement site M.

Some pulse waves generated by the heartbeat of the subject directly come to the measurement site M, and some are reflected by other sites than the measurement site M and indirectly come to the measurement site M. For example, as described above, when the wrist of the subject is the measurement site M, the pulse wave passing through the measurement site M and reflected by the finger tip and, for example, the pulse wave reflected near the thigh reach the measurement site M as indirect waves delayed with respect to the direct waves directly reaching the measurement site M. Therefore, in the waveform of the detection signal S1, a peak derived from the direct wave and a peak derived from the indirect wave are supposed to be observed almost simultaneously.

On the other hand, the capillaries exist near the epidermis of the measurement site M. The illumination light radiated from the light emitting part 32 to the measurement site M is absorbed by the blood within the capillaries. That is, the blood within the capillaries acts to reduce the light reception intensity by the light receiving part 36 (to reduce the S/N-ratio of the detection signal S1). Therefore, in consideration of the absorption of the illumination light by the blood within the capillaries, the distinction between the direct wave and the indirect wave in the detection signal S1 is obscure as shown by the waveform WL in FIG. 3. However, in the state in which the detection surface 16 of the casing part 12 is pressed on the measurement site M into close contact, the capillaries of the measurement site M collapse by the pressure, and the blood is excluded as a result (the skin turns whitish by the pressure). That is, the absorption of light by the blood within the capillaries is reduced, and consequently, the reduction of the light reception intensity is suppressed. Therefore, in the state in which the measurement site M and the detection surface 16 are sufficiently in close contact, the distinction between the peak P1 derived from the direct wave (an exemplification of a first peak) and the peak P2 derived from the indirect wave (an exemplification of a second peak) is clear as shown by the waveform WH and the waveform WM in FIG. 3. It is estimated that the reason that the difference in waveform of the detection signal S1 is observed depending on the contact condition between the measurement site M and the detection surface 16 is as described above.

On the background of the above described knowledge, the irradiation control unit 42 of the first embodiment controls whether or not to allow laser irradiation by the laser irradiation part 34 according to the waveform of the detection signal S1. Specifically, when the peak P1 and the peak P2 almost simultaneously exist in the detection signal S1 within a predetermined time τ, the state in which the measurement site M and the detection surface 16 are sufficiently in close contact (in other words, the laser does not leak) may be estimated, and thus, the irradiation control unit 42 allows laser irradiation by the laser irradiation part 34. The peak P1 is a peak at which the signal value of the detection signal S1 becomes the maximum due to the direct wave and the peak P2 is a peak generated immediately after the peak P1 due to the indirect wave. On the other hand, when two peaks do not exist in the detection signal S1 within the predetermined time τ, it may be estimated that the contact between the measurement site M and the detection surface 16 is insufficient (in other words, the laser may leak) and thus, the irradiation control unit 42 prohibits laser irradiation by the laser irradiation part 34.

The interval between the peak P1 and the peak P2 of the detection signal S1 is considered. The velocity of the pulse wave propagating in the blood vessel is generally from 5 m/s to 10 m/s. The distance from the wrist as the measurement site M to the finger tip is about 10 cm at most, and the time after the pulse wave passes through the measurement site M and before the pulse wave reflected by the finger tip reaches the measurement site M as the indirect wave is about from 0.01 seconds to 0.02 seconds. Further, the distance from the wrist as the measurement site M to the thigh is about 200 cm at most, and the time of about 0.4 seconds may lapse at the maximum after the direct wave reaches the wrist as the measurement site M and before the indirect wave reflected by the thigh reaches the measurement site M. That is, the peak P2 derived from the indirect wave of the detection signal S1 reaches the measurement site M with a delay from 0.01 seconds to 0.4 seconds from the peak P1 derived from the direct wave. In consideration of the above described situation, in the first embodiment, the time τ assumed as the interval between the peak P1 and the peak P2 is set to 0.4 seconds (more preferably 0.2 seconds)

FIG. 4 shows transitions of first-order differential values with respect to the signal values of the detection signal S1. Like FIG. 3, the transitions of the differential values are shown with respect to a plurality of cases in which the contact condition between the detection surface 16 and the measurement site M is varied (pressing force: large/medium/zero). As described above, in the state in which the measurement site M and the detection surface 16 are sufficiently in close contact, a minimum point L corresponding to the recessed portion between the peak P1 and the peak P2 of the detection signal S1 is observed within the range of the predetermined time τ from the peak Q of the differential value. In consideration of the above described tendency, whether or not to allow the laser irradiation by the laser irradiation part 34 can be controlled according to whether or not the minimum point L exists within the range of the predetermined time T from the peak Q of the differential value of the signal value of the detection signal S1. Specifically, if the minimum point L exists within the predetermined time τ from the peak Q of the differential value of the detection signal S1, the state in which the measurement site M and the detection surface 16 are sufficiently in close contact may be estimated, and the irradiation control unit 42 allows the laser irradiation by the laser irradiation part 34. On the other hand, if the minimum point L does not exist within the predetermined time τ from the peak Q of the differential value of the detection signal S1, the state in which the measurement site M and the detection surface 16 are not sufficiently in close contact may be estimated, and the irradiation control unit 42 prohibits the laser irradiation by the laser irradiation part 34.

### Biological Information Acquisition Method

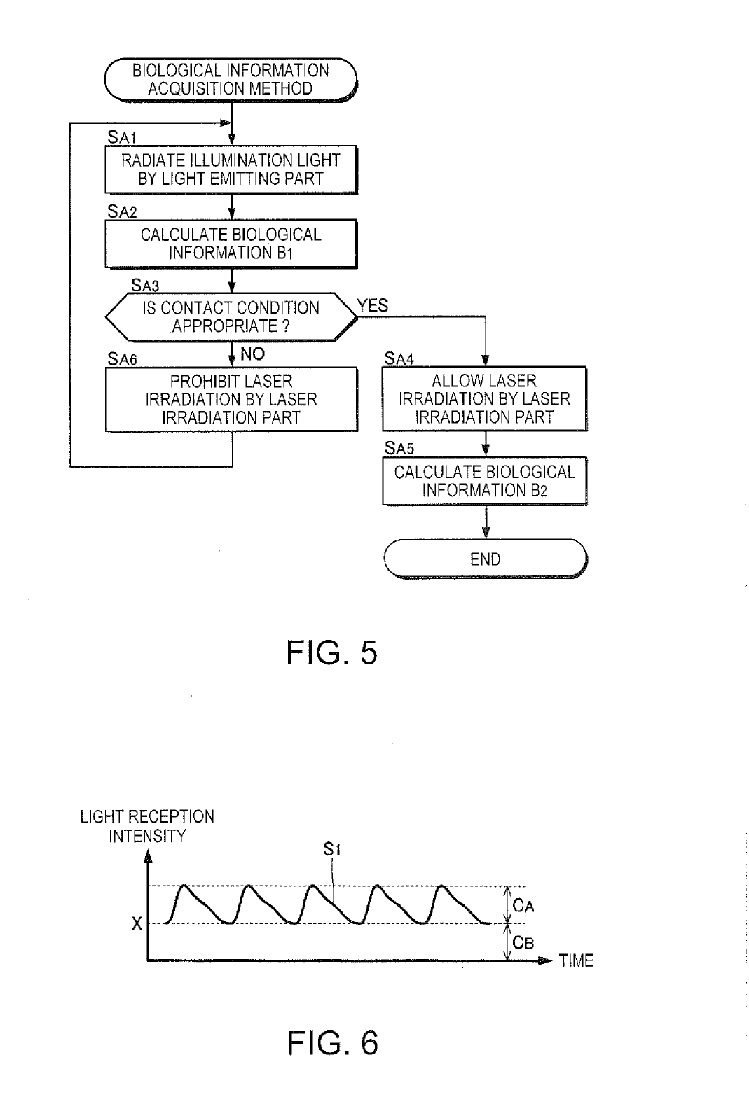

An operation method of the above exemplified biological information acquisition apparatus 100 (biological information acquisition method) will be explained. FIG. 5 is a flowchart of the biological information acquisition method of the first embodiment. For example, the processing in FIG. 5 is started when an instruction of starting a measurement is given to an input device (not shown).

When the biological information acquisition method in FIG. 5 is started, the irradiation control unit 42 controls the light emitting part 32 to radiate the illumination light (SA1). The analytical processing unit 44 acquires the detection signal S1 generated by the light receiving part 36 (light receiving element 361) when the illumination light is radiated and analyzes the detection signal S1 to calculate the biological information B1 including the pulse wave (SA2). A known technique may be optionally employed for the calculation of the biological information B1 corresponding to the detection signal S1. The biological information B1 calculated by the analytical processing unit 44 is displayed on the display device 26.

The biological information B1 is calculated in the above described procedure, then, the irradiation control unit 42 determines whether or not the contact condition between the measurement site M and the detection surface 16 is appropriate (SA3). In other words, the unit determines whether or not the measurement site M and the detection surface 16 are sufficiently in close contact to the degree that prevents leakage of the laser. Specifically, as described above, the irradiation control unit 42 of the first embodiment determines whether or not the peak P2 exists within the predetermined time τ from the peak P1 of the detection signal S1 (or whether or not the minimum point L exists within the predetermined time τ from the peak Q of the differential value of the detection signal S1).

If the peak P2 exists within the time τ from the peak P1 of the detection signal S1 (SA3: Yes), in other words, if the measurement site M and the detection surface 16 are sufficiently in close contact, the irradiation control unit 42 allows laser irradiation by the laser irradiation part 34 (SA4). Specifically, the irradiation control unit 42 controls the laser irradiation part 34 to radiate the laser. The measurement site M and the detection surface 16 are sufficiently in close contact without a gap, and thus, leakage of the laser to the outside is avoided. The analytical processing unit 44 acquires the detection signal S2 generated by the light receiving part 36 (light receiving element 362) when the laser is radiated and analyzes the detection signal S2 to calculate the biological information B2 including the blood flow velocity (SA5). A known technique may be optionally employed for the calculation of the biological information B2 corresponding to the detection signal S2. The biological information B2 calculated by the analytical processing unit 44 is displayed on the display device 26.

On the other hand, if the peak P2 does not exist within the time T from the peak P1 of the detection signal S1 (SA3: No), in other words, if the measurement site M and the detection surface 16 are not sufficiently in close contact, the irradiation control unit 42 prohibits laser irradiation by the laser irradiation part 34 (SA6). That is, the laser irradiation by the laser irradiation part 34 (SA4) and the calculation of the biological information B2 by the analytical processing unit 44 (SA5) are not executed. The processing moves to step SA1 under the condition that the irradiation control unit 42 prohibits the laser irradiation. Therefore, the irradiation of the illumination light by the light emitting part 32 (SA1), the calculation of the biological information according to the detection signal S1 (SA2), and the determination of the contact condition between the measurement site M and the detection surface 16 (SA3) are repeated. Note that the processing is moved to step SA3 after the laser irradiation is prohibited, and thereby, the repetition of the irradiation of the illumination light (SA1) and the calculation of the biological information E1 (SA2) can be omitted.

The subject adjusts the position of the casing part 12 and the length of the belt 14 and the state is moved to the state in which the measurement site M and the detection surface 16 are sufficiently in close contact, the determination result at step SA3 shifts to affirmation (SA3: Yes), and the laser irradiation of the measurement site M (SA4) and the calculation of the biological information B2 according to the detection signal S2 (SA5) are executed. Note that, if the determination result at step SA3 is denial, a message for giving an instruction of adjustment of the contact condition between the measurement site M and the detection surface 16 to the subject can be displayed on the display device 26.

As described above, in the first embodiment, whether or not to allow the laser irradiation by the laser irradiation part 34 is controlled according to the detection signal S1 indicating the light reception intensity when the illumination light is radiated by the light emitting part 32, and thereby, the biological information B2 according to the detection signal S2 can be acquired with the reduced possibility of the erroneous laser irradiation. In the first embodiment, particularly, the detection signal S1 is used for both the acquisition of the biological information B1 and the control as to whether or not to allow the laser irradiation. Therefore, there is an advantage that the configuration of the biological information acquisition apparatus 100 is simplified compared to a configuration in which a light emitting part for acquisition of the biological information B1 and a light emitting part for control as to whether or not to allow the laser irradiation are separately provided.

Further, in the first embodiment, whether or not to allow the laser irradiation is controlled according to the waveform of the detection signal S1, and therefore, there is an advantage that whether or not to allow the laser irradiation may be appropriately controlled by the simple processing of analyzing the waveform of the detection signal S1. Specifically, whether or not to allow the laser irradiation is controlled according to whether or not the peak P2 exists within the predetermined time τ from the peak P1 of the detection signal S1, and therefore, the laser irradiation can be allowed only when the measurement site M and the detection surface 16 are in close contact to the degree that excludes the blood within the capillaries.

### Second Embodiment

A second embodiment of the invention will be explained. In the first embodiment, whether or not the contact condition between the measurement site M and the detection surface 16 is appropriate is determined by the analysis of the waveform of the detection signal S1 generated by the light receiving part 36 when the illumination light is radiated. The irradiation control unit 42 of the second embodiment controls whether or not to allow the laser irradiation by the laser irradiation part 34 according to the light reception intensity indicated by the detection signal S1. Note that, in the respective forms to be exemplified as below, the signs referred to in the explanation of the first embodiment are also used for the elements having the same actions and functions as those of the first embodiment and their detailed explanation will be omitted as appropriate.

FIG. 6 is a waveform chart of the detection signal S1 generated by the light receiving part 36 when the measurement site M is irradiated with the illumination light. As understood from FIG. 6, the detection signal S1 contains a fluctuating component CA and a stationary component CB. The fluctuating component CA is a pulsating component that periodically fluctuates due to the heartbeat of the subject. On the other hand, the stationary component CB is a temporally stationary component (direct-current component).

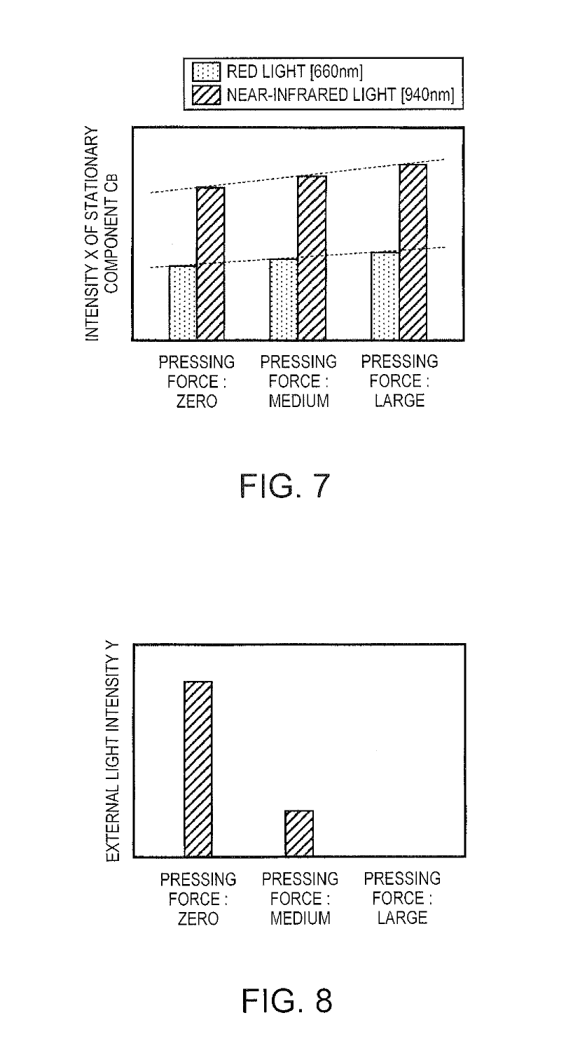

FIG. 7 shows results of measurements of intensity X of the stationary component CB of the detection signal S1. FIG. 7 shows the intensity X of the stationary component CB with respect to a plurality of cases in which the contact condition of the detection surface 16 with the measurement site M is varied (pressing force: large/medium/zero). Further, in FIG. 7, the case where the measurement site M is irradiated with red light having a wavelength of 660 nm as illumination light and the measurement site M is irradiated with near-infrared light having a wavelength of 940 nm as illumination light are assumed.

A tendency that, regardless of the wavelength of the illumination light, as the pressing force of the detection surface 16 on the measurement site M increases (the degree of close contact between the measurement site M and the detection surface 16 is higher), the intensity X of the stationary component CB of the detection signal S1 increases may be confirmed from FIG. 7. The above described tendency is observed because, as described in the first embodiment, as the pressing force on the measurement site M increases, the capillaries of the measurement site M collapse and the blood is excluded, and the absorption of light by the blood within the capillaries is reduced.

On the background of the above described knowledge, the irradiation control unit 42 of the second embodiment controls whether or not to allow laser irradiation by the laser irradiation part 34 according to the intensity X of the stationary component CB of the detection signal S1. Specifically, at the step SA3 of the biological information acquisition method in FIG. 5, the irradiation control unit 42 determines whether or not the intensity X of the stationary component CB is larger than a threshold value XTH. The threshold value XTH is selected experimentally or statistically to be approximately equal to the intensity X of the stationary component CB observed when the measurement site M and the detection surface 16 are sufficiently in close contact. Specifically, the threshold value XTH is set in adjustment processing before use of the biological information acquisition apparatus 100. In the adjustment processing, as is the case of the exemplification of the first embodiment, whether or not the peak P2 exists within the predetermined time τ from the peak P1 of the detection signal S1 is determined. Then, the intensity X of the stationary component CB in the state in which the peak P1 and the peak P2 are observed within the time τ (i.e. the state in which the measurement site M and the detection surface 16 are sufficiently in close contact) is stored as the threshold value XTH in the memory device 24. A representative value of the intensity X in the plurality of times of adjustment processing (e.g. an average value) can be employed as the threshold value XTH.

If the intensity X of the stationary component CB is larger than the threshold value XTH (X>XTH), the state in which the measurement site M and the detection surface 16 are sufficiently in close contact may be estimated, and the the irradiation control unit 42 allows laser irradiation by the laser irradiation part 34 (SA4). On the other hand, if the intensity X of the stationary component CB is smaller than the threshold value XTH (X<XTH), the state in which the measurement site M and the detection surface 16 are not sufficiently in close contact may be estimated, and the irradiation control unit 42 prohibits laser irradiation by the laser irradiation part 34 (SA5). The rest of the configuration and the operation are the same as those of the first embodiment.

As described above, in the second embodiment, whether or not to allow the laser irradiation by the laser irradiation part 34 is controlled according to the detection signal S1 indicating the light reception intensity when the illumination light is radiated by the light emitting part 32, and thereby, the possibility of erroneous laser irradiation can be reduced as is the case of the first embodiment. In the second embodiment, particularly, whether or not to allow the laser irradiation is controlled according to the light reception intensity indicated by the detection signal S1, and therefore, there is an advantage that whether or not to allow the laser irradiation may be appropriately controlled by the simple processing of analyzing the detection signal S1. Specifically, whether or not to allow the laser irradiation is controlled according to the intensity X of the stationary component CB of the detection signal S1, and therefore, the measurement site M can be irradiated with the laser only when the measurement site M and the detection surface 16 are in close contact to the degree that excludes the blood within the capillaries.

### Third Embodiment

An external light from the sun or lighting equipment may enter between the measurement site M and the detection surface 16. Therefore, even when both the light emitting part 32 and the laser irradiation part 34 are turned off, the light receiving part 36 may receive the external light. FIG. 8 shows results of measurements of the light reception intensity (hereinafter, referred to as “external light intensity”) Y of the light receiving part 36 in the condition in which both the light emitting part 32 and the laser irradiation part 34 are turned off. FIG. 8 shows the external light intensity Y with respect to a plurality of cases in which the contact condition of the detection surface 16 with the measurement site M is varied (pressing force: large/medium/zero). A tendency that the external light intensity Y depends on the contact condition between the measurement site M and the detection surface 16 may be confirmed from FIG. 8. Specifically, as the pressing force of the detection surface 16 on the measurement site M increases (the degree of close contact between the measurement site M and the detection surface 16 is higher), the external light intensity Y decreases.

On the background of the above described knowledge, the irradiation control unit 42 of the third embodiment controls whether or not to allow laser irradiation by the laser irradiation part 34 according to the light reception intensity (external light intensity Y) of the light receiving part 36 when the light emitting part 32 is turned off. Specifically, at the step SA3 of the biological information acquisition method in FIG. 5, the irradiation control unit 42 determines whether or not the external light intensity Y indicated by the detection signal S generated by the light receiving part 36 after the light emitting part 32 is turned off is smaller than a threshold value YTH. The threshold value YTH is set to a predetermined value (e.g. a positive number near zero) close to the external light intensity Y observed when the measurement site M and the detection surface 16 are sufficiently in close contact. The external light intensity Y is the light reception intensity of one of the light receiving element 361 and the light receiving element 362 or an average of the light reception intensity of both the light receiving element 361 and the light receiving element 362 in the light receiving part 36.

If the external light intensity Y is smaller than the threshold value YTH (Y<YTH), the state in which the measurement site M and the detection surface 16 are sufficiently in close contact may be estimated, and the irradiation control unit 42 allows laser irradiation by the laser irradiation part 34 (SA4). On the other hand, if the external light intensity Y is larger than the threshold value YTH (Y>YTH), the state in which the measurement site M and the detection surface 16 are not sufficiently in close contact may be estimated, and the the irradiation control unit 42 prohibits laser irradiation by the laser irradiation part 34 (SA5). The rest of the configuration and the operation are the same as those of the first embodiment.

As described above, in the third embodiment, whether or not to allow the laser irradiation by the laser irradiation part 34 is controlled according to the light reception intensity (external light intensity Y) of the light receiving part 36 when the light emitting part 32 is turned off, and thereby, the possibility of erroneous laser irradiation can be reduced as is the case of the first embodiment. In the third embodiment, particularly, the light reception intensity of the light receiving part 36 when the light emitting part 32 is turned off is used for the control of the laser irradiation, and therefore, there is an advantage that the laser irradiation may be appropriately controlled by the simpler processing than those of the first embodiment and the second embodiment.

### MODIFIED EXAMPLES

The respective embodiments exemplified as above may be variously modified. The specific modified forms will be exemplified as below. Two or more forms arbitrarily selected from the following exemplifications can be appropriately combined.

(1) In the above described respective embodiments, the wristwatch-type biological information acquisition apparatus 100 is exemplified, however, the form of the biological information acquisition apparatus 100 is not limited to the exemplification. For example, the biological information acquisition apparatus 100 can be realized in arbitrary forms including forms similar to accessories (e.g. bracelet-type, necklace-type, earring-type), spectacle-type, sticker-type to be attached to the measurement site M of the subject. Further, in the above description, the portable biological information acquisition apparatus 100 that can be worn on the body of the subject is exemplified, however, the biological information acquisition apparatus can be realized as a stationary measurement apparatus.

(2) In the above described respective embodiments, the pulsebeat is estimated as the biological information B1 and the blood flow velocity and the blood pressure are estimated as the biological information B2, however, the biological information B is not limited to the exemplifications. For example, various blood component concentrations including blood glucose concentration and hemoglobin concentration, blood oxygen concentration, and neutral fat concentration can be calculated as the biological information. B1 or biological information B2. Further, in the above described respective embodiments, the biological information B2 is calculated by the analysis of the detection signal S2, however, the biological information B2 can be calculated (SA5) from both the detection signal S1 and the detection signal S2. The calculation of the biological information B1 can be omitted.

(3) The biological information acquisition apparatus 100 exemplified in the above described respective embodiments may be realized by cooperation of the control device 22 and the programs as described above. A program according to a preferred embodiment of the invention allows a computer connected to a detection device 28 including a light emitting part 32 that irradiates a measurement site M of a living organism with a non-laser illumination light, a laser irradiation part 34 that irradiates the measurement site M with a laser, and a light receiving part 36 that receives lights from the measurement site M and generates a detection signal S1 indicating light reception intensity when the illumination light is radiated and a detection signal S2 indicating light reception intensity when the laser is radiated to function as an irradiation control unit 42 that controls whether or not to allow laser irradiation by the laser irradiation part 34 according to the detection signal S1 and an analytical processing unit 44 that acquires biological information B2 according to the detection signal S2. The above exemplified program may be provided in a form stored in a computer-readable recording medium and installed in the computer. The recording medium is e.g. a non-transitory recording medium and including an optical recording medium (optical disk) such as a CD-ROM as a good example, and may include recording media in known arbitrary formats such as a semiconductor recording medium and a magnetic recording medium. Further, the program can be delivered in a form of delivery via a communication network.

The entire disclosure of Japanese Patent Application No. 2015-248013 is hereby incorporated herein by reference.

What is claimed is:

1. A biological information acquisition apparatus comprising:
a light emitting part provided on a detection surface facing a measurement site and irradiating the measurement site with a non-laser illumination light;
a laser irradiation part provided on the detection surface and irradiating the measurement site with a laser;
a light receiving part provided on the detection surface and receiving lights from the measurement site, and generating a first detection signal indicating light reception intensity when the illumination light is radiated and a second detection signal indicating light reception intensity when the laser is radiated;
an irradiation control unit that controls whether or not to allow laser irradiation by the laser irradiation part according to the first detection signal; and
an analytical processing unit that acquires biological information according to the second detection signal.

2. The biological information acquisition apparatus according to claim 1, wherein the analytical processing unit acquires biological information according to the first detection signal and biological information according to the second detection signal.

3. The biological information acquisition apparatus according to claim 1, wherein the first detection signal is a pulse wave signal containing a pulsation component of an artery of the measurement site, and
the irradiation control unit controls whether or not to allow laser irradiation by the laser irradiation part according to a waveform of the first detection signal.

4. The biological information acquisition apparatus according to claim 3, wherein the irradiation control unit allows laser irradiation by the laser irradiation part if a peak different from a first peak exists within a predetermined time from the first peak at which a signal value of the first detection signal becomes the maximum.

5. The biological information acquisition apparatus according to claim 1, wherein the irradiation control unit controls whether or not to allow laser irradiation by the laser irradiation part according to light reception intensity indicated by the first detection signal.

6. The biological information acquisition apparatus according to claim 5, wherein the irradiation control unit controls whether or not to allow laser irradiation according to intensity of a stationary component in the first detection signal.

7. A biological information acquisition apparatus comprising:
a light emitting part provided on a detection surface facing a measurement site and irradiating the measurement site with a non-laser illumination light;
a laser irradiation part provided on the detection surface and irradiating the measurement site with a laser;
a light receiving part provided on the detection surface and receiving lights from the measurement site, and generating a first detection signal indicating light reception intensity when the illumination light is radiated and a second detection signal indicating light reception intensity when the laser is radiated;
an irradiation control unit that controls whether or not to allow laser irradiation by the laser irradiation part according to light reception intensity of the light receiving part when the light emitting part is turned off; and
an analytical processing unit that acquires biological information according to the first detection signal and the second detection signal.

8. A biological information acquisition method by a biological information acquisition apparatus, comprising:
irradiating a measurement site with a non-laser illumination light from a detection surface facing the measurement site;
controlling whether or not to allow laser irradiation to the measurement site according to a first detection signal indicating light reception intensity of a light received by a light receiving part provided on the detection surface when the illumination light is radiated;
if the laser radiation is allowed, irradiating the measurement site with a laser from a laser irradiation part provided on the detection surface; and
acquiring biological information according to a second detection signal indicating light reception intensity of a light received from the measurement site by the light receiving part when the laser is radiated.
